(19) United States
(12) Patent Application Publication
(10) Pub. No.: US 2007/0238902 A1
(43) Pub. Date: Oct. 11, 2007

(54) Method for Performing Aldol Reaction in Water

(76) Inventors: Shu Kobayashi, Tokyo (JP); Kei Manabe, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 11/587,430

(22) PCT Filed: Feb. 14, 2005
(86) PCT No.: PCT/JP05/02657

§ 371 Date: Jan. 12, 2007

(30) Foreign Application Priority Data
Apr. 26, 2004 (JP) 2004-130242

(51) Int. Cl. C07C 45/72; C07B 61/00; C07C 49/747; C07C 49/835; C07C 49/84; C07C 49/83; C07C 327/22

(52) U.S. Cl. 568/75; 568/391

(57) ABSTRACT

An aldol reaction in an aqueous solution between an aldehyde represented by the following formula (I):
R—CHO (I)
(wherein R is a hydrocarbon group which may have an substituent), and a silicon enolate represented by the following formula (II):

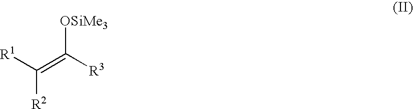

(wherein R¹ and R² are a hydrogen atom or an aliphatic hydrocarbon group, at least one of them being an aliphatic hydrocarbon group; and

R³ is a substituent selected from the group consisting of an aliphatic hydrocarbon group, an aromatic hydrocarbon group and a sulfur-containing substituent, wherein R² and R³ may be combined with each other to form a ring) is performed so as to generate diastereoselectivity by using FeCl₃, which is a low-cost Lewis acid, and a surfactant.

### TECHNICAL FIELD

The present invention relates to a method for performing an aldol reaction in water. The invention more particularly relates to a method for reacting an aldehyde and a silicon enolate with each other in water in the presence of FeCl₃ and a surfactant and, then, obtaining a product with a high diastereoselectivity.

### BACKGROUND ART

In recent years, a method for performing an organic synthesis reaction in water without using a toxic organic solvent has been attracting attention from the viewpoint of environmental protection (Non-patent documents 1 to 3). To date, it has been reported that a Lewis acid catalyst in which a rare earth metal such as scandium or ytterbium and an anionic surfactant are combined with each other (Non-patent documents 4 to 7) effectively catalyzes an aldol reaction (Non-patent documents 4 to 9), an allylation reaction (Non-patent document 10), a Michael reaction (Non-patent document 11) or the like in water. Although these reactions are advantageous in that they proceed with a high stereoselectivity and a high yield, they are disadvantageous in that, since the rare-earth metal is high in cost, reactions using such catalyst system as described above are likely to be high in cost.

On the other hand, the aldol reaction which gives a β-hydroxycarbonyl compound by the reaction in which a metal enolate or an enol derivative is added to a carbonyl compound has been known as an important method for forming a carbon-carbon bond and has widely been used also for synthesizing natural substances. When the aldol reaction can be performed in water at low cost and, thereby obtaining a product with a high diastereoselectivity, a cost reduction of the reaction can be realized, and it is expected that there will be applications thereof for synthesis of natural substances on an industrial scale.

Therefore, development of a Lewis acid catalyst which shows high catalytic activity even in water and is lower in cost and a method for performing an aldol reaction with a high diastereoselectivity in water by using the Lewis acid catalyst has been desired.

Non-patent document 1: C.-J. Li and T.-H. Chan, “Organic Reactions in Aqueous Media”, John Wiley & Sons, New York (1997).

Non-patent document 2: P. A. Grieco “Organic Synthesis in Water”, Brackie Academic and Professional, London (1998).

Non-patent document 3: S. Kobayashi and K. Manabe, Acc. Chem. Res. 35, 209 (2002).

Non-patent document 4: K. Manabe, Y. Mori, T. Wakabayashi, S. Nagayama, and S. Kobayashi, J. Am. Chem. Soc. 122. 7202 (2000).

Non-patent document 5: S. Kobayashi and T. Wakabayashi, Tetrahedron Lett., 39, 5389 (1998).

Non-patent document 6: K. Manabe, Y. Mori and S. Kobayashi, Tetrahedron, 55, 11203 (1999).

Non-patent document 7: K. Manabe and S. Kobayashi, Synlett. 1999. 547.

Non-patent document 8: Y. Mori, K. Manabe and S. Kobayashi, Angew. Chem. Int. Ed., 40, 2816 (2001).

Non-patent document 9: Y. Mori, J. Kobayashi, K. Manabe, and S. Kobayashi, Tetrahedron, 58, 8263 (2002)

Non-patent document 10: S. Kobayashi, T. Wakabayashi, and H. Oyamada, Chem. Lett., 1997, 831.

Non-patent document 11: Y. Mori, K. Kakumoto, K. Manabe, and S. Kobayashi, Tetrahedron Lett., 41, 3107 (2000).

Non-patent document 12: S. Kobayashi, S. Nagayama, and T. Busujima, J. Am. Chem. Soc., 120, 8287 (1998).

Non-patent document 13: E. P. Kundig and C. M. Saudan “Lewis acids in Organic Synthesis” ed. by H. Yamamoto, Wiley-VCH, Weinheim (2000), Vol. 2, Chap. 14, p. 597.

Non-patent document 14: O. Munoz-Muniz, M. Quintanar-Audelo, and E. Juaristi, J. Org. Chem. 88, 1622 (2003).

Accordingly, the invention has been accomplished in view of the above-described circumstances and an object of the invention is to solve the problems of the prior arts and to provide a method for performing an aldol reaction in water with a high diastereoselectivity at a low cost.

### DISCLOSURE OF INVENTION

As a means to solve the above problems, the invention firstly provides a method for performing an aldol reaction in water which is characterized in that an aldehyde represented by the following formula (I):
R—CHO (I)
(wherein R represents a hydrocarbon group which may have a substituent), and a silicon enolate represented by the following formula (II): 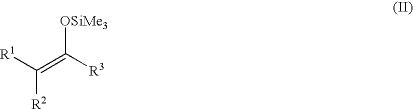
(wherein R¹ and R² each are a hydrogen atom or an aliphatic hydrocarbon group, at least one of them being an aliphatic hydrocarbon group; and R³ represents a substituent selected from the group consisting of an aliphatic hydrocarbon group, an aromatic hydrocarbon group and a sulfur-containing substituent, wherein R² and R³ may be combined with each other to form a ring) are allowed to react with each other in water in the presence of FeCl₃ and a surfactant.

Secondly, the invention provides a method for performing the aldol reaction in water in which the surfactant is sodium alkyl sulfonate. Thirdly, the invention provides the method for performing the aldol reaction in water, in which the surfactant is sodium dodecyl sulfonate. Fourthly, the invention provides the method for performing the aldol reaction in water, in which the surfactant is sodium benzenesulfonate having a long-chain alkyl group. Fifthly, the invention provides the method for performing the aldol reaction in water, in which the surfactant is sodium octylbenzenesulfonate or sodium dodecylbenzene sulfonate.

The invention sixthly provides any one of the above-described methods for performing the aldol reaction in water, in which the reaction is performed in the coexistence with a base. Seventhly, the invention provides the method for performing the aldol reaction in water, in which the base is sodium hydroxide.

### BEST MODE FOR CARRYING OUT THE INVENTION

A method for performing an aldol reaction in water according to the invention reacts an aldehyde and a silicon enolate with each other in water with a diastereoselectivity and is characterized in that FeCl₃ and a surfactant are used as a catalyst system.

It has been considered to date that FeCl₃ which is used as a catalyst in the method for performing the aldol reaction in water according to the invention is not suited for use with water (it has no or low catalyst activity in water) (Non-patent document 12). As a result of an intense study, the present inventors have found that by having a surfactant present with the FeCl₃, the aldol reaction in water progresses with a high yield and a high diastereoselectivity, thereby accomplishing the invention.

Further, the method for performing the aldol reaction in water according to the invention is advantageous compared with a conventional method for performing the aldol reaction in water using a rare-earth metal compound as a catalyst in that FeCl₃ is not only a compound exhibiting a strong Lewis acidity (Non-patent document 13) but also easily available at an extremely low cost (for example, at a price of about one 100th or less of the price of Sc(OTf)₃ per gram).

In the method for performing the aldol reaction in water according to the invention, the aldehyde is represented by the following formula (I):
R—CHO (I)

In the formula (I), R represents a hydrocarbon group which may have a substituent. Specific examples of such hydrocarbon groups include alkyl groups such as a methyl group, an ethyl group, an n-propyl group, an i-propyl group, an n-butyl group, a sec-butyl group and a tert-butyl group; alkenyl groups such as a vinyl group, a propenyl group and a butenyl group; alkynyl groups such as an ethynyl group and a propynyl group; and aryl groups such as a phenyl group and a tolyl group. These groups may each further be substituted with an aromatic hydrocarbon group, a halogen group, an alkoxy group or the like.

On the other hand, the silicon enolate is represented by the following formula (II):

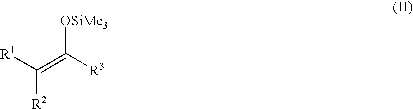

In the formula (II), R¹ and R² are a hydrogen atom or an aliphatic hydrocarbon group, at least one of them being an aliphatic hydrocarbon group.

Examples of such aliphatic hydrocarbon groups include alkyl groups such as a methyl group, an ethyl group, an n-propyl group, an i-propyl group, an n-butyl group, a sec-butyl group and a tert-butyl group. Further, in the formula (II), R³ represents a substituent selected from the group consisting of an aliphatic hydrocarbon group, an aromatic hydrocarbon group and a sulfur-containing substituent. Specifically, alkyl groups such as a methyl group, an ethyl group, an n-propyl group, an i-propyl group, an n-butyl group, a sec-butyl group and a tert-butyl group; aryl groups such as a phenyl group, a tolyl group and a naphthyl group; and sulfur-containing substituents such as an alkylthio group and an arylthio group are usable. These groups, R² and R³, may further be combined with each other, to thereby form an aliphatic cyclic group.

The research by the present inventors has revealed that the method for performing the aldol reaction in water exhibits a particularly high diastereoselectivity when, in the formula (II) which represents the silicon enolate, R¹ represents an alkyl group and R² represents a hydrogen atom.

In the method for performing the aldol reaction according to the invention as described above, the surfactant which constitutes the catalyst system together with FeCl₃ may be any type and is not particularly limited. For example, an alkali metal salt of an alkyl sulfonic acid, a metallic salt of an alkylbenzenesulfonic acid, cetyltrimethylammonium bromide (CTAB) and TRITON® (trademark) X-100 and the like are applicable. Among these surfactants, an anionic surfactant is preferred and, specifically, an alkali metal salt of an alkyl sulfonic acid such as sodium dodecyl sulfonate and an alkali metal salt of an alkylbenzenesulfonic acid such as sodium octylbenzenesulfonate or sodium dodecylbenzenesulfonate are favorably illustrated.

The amount of any one of these surfactants to be added is not particularly limited, but, for example, it can be 1 to 100 mol % of the aldehyde which is the reaction substrate.

In the method for performing the aldol reaction in water according to the invention, for the purpose of preventing the silicon enolate from being hydrolyzed and, thus further enhancing reaction yield, a base may be introduced in the reaction system. Types of such bases are not particularly limited and, for example, NaOH, KOH, imidazole and triethylamine are illustrated. Among these bases, NaOH is preferable. Further, the amount of the base to be added is not particularly limited but is preferably in the range of from 1 to 10 mol % of the aldehyde which is the reaction substrate. Further, in the method for performing the aldol reaction according to the invention, the reaction solvent is water. As described in embodiments below, the method for performing the aldol reaction in water according to the invention exhibits a high yield in a system in which water only is the reaction solvent, higher than in the presence of an organic solvent.

Hereinafter, embodiments of the invention will be described in more detail with reference to the following Examples. It goes without saying that the invention is not restricted to these Examples and various embodiments may be considered.

### EXAMPLES

### Example 1

Designating a reaction of benzaldehyde and the silicon enolate (Compound 1) as shown in the following reaction formula (A): 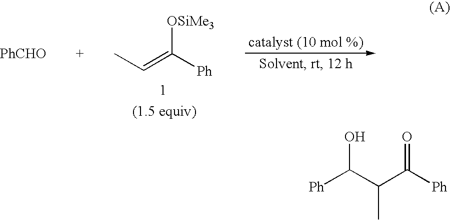
as a model reaction, the catalytic capability of various types of metallic salts were compared with one another in the presence of sodium dodecyl sulfonate (hereinafter, referred to also as “SDS”).

In the reaction of the formula (A), since the hydrolysis reaction of the silicon enolate competes with the desired aldol reaction, the hydrolysis reaction is considered to be appropriate as an indicator in screening catalysts for the aldol reaction. It was confirmed that, even compared with various types of metallic salts, FeCl₃ showed an appropriate yield and gives a product with a higher syn/anti selectivity than Sc(OTf)₃ which often is used

TABLE 1

| | | | Yield | |
|---|---|---|---|---|
| Reaction | Catalyst (mol %) | Solvent | % | Syn/anti |
| 1 | Sc(OTf)₃(10) + SDS(30) | H₂O | 95 | 50/50 |
| 2 | FeCl₃(10) + SDS(30) | H₂O | 53 | 85/15 |
| 3 | FeCl₃(10) | H₂O/THF = 1/9 | 45 | 71/29 |

Further, it was confirmed that the reaction in water/surfactant showed higher yield and diastereoselectivity than the reaction in the water/THF mixed solvent. It is considered that this is because transfer of protons comprising the hydrolysis reaction of the silicon enolate (1) is suppressed in a heterogeneous system of water/surfactant.

### Example 2

Then, respective diastereoselectivities in the cases where FeCl₃ and Sc(OTf))₃ were used as the catalyst under the conditions as shown in the following reaction formula (B):

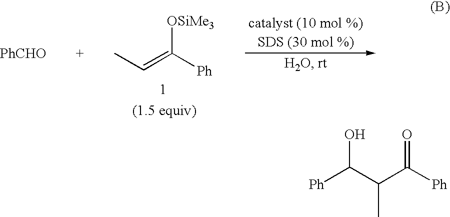

were compared with one another (Table 2).

TABLE 2

| | | |
|---|---|---|
| Reaction time | Sc(OTf)₃ | FeCl₃ |

| (min) | Yield % | Syn/anti | Yield % | Syn/anti |
|---|---|---|---|---|
| 10 | 23 | 51/49 | 46 | 86/14 |
| 60 | 74 | 50/50 | 57 | 86/14 |
| 720 | 95 | 50/50 | 53 | 85/15 |

Jauristi et al. have described that, in the aldol reaction in an aqueous solvent using CeCl₃ (H₂O/i-PrOH=1/19), epimerization of the product was observed (Non-patent document 14). However, in the reactions using FeCl₃ according to the invention, and using the Sc(OTf)₃ reported previously by the present inventors as catalysts, a change in the diastereoselectivity with the passage of time was not noticed. Further, even when the product in which a diastereomer ratio was enhanced was directly treated in these catalyst systems, a change in the diastereomer ratio was not noticed (Table 3).

TABLE 3

Syn/anti

| Reaction | Catalyst | Before reaction | After reaction |
|---|---|---|---|
| 1 | Sc(OTf)₃ | 84/16 | 82/18 |
| 2 | FeCl₃ | 84/16 | 82/18 |

From these results, it was confirmed that, in the aldol reaction in water using these catalysts, the epimerization did not occur.

### Example 3

Next, in order to optimize the aldol reaction in water using FeCl₃ as the catalyst, the effects of various surfactant types were studied. The results are shown in Table 4.

TABLE 4

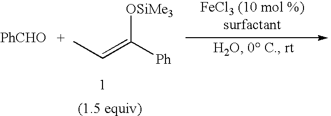

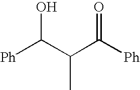

| Reaction | Surfactant (mol %) | Yield (%) | Syn/anti |
|---|---|---|---|
| 1 | SDS (30) | 71 | 84/16 |
| 2 | SDS (30) | 81 | 86/14 |
| 3 | SDS (10) | 83 | 87/13 |
| 4 | SDS (5) | 71 | 87/13 |
| 5 | — | Trace | 87/13 |
| 6 | CTAB | 21 | 92/8 |
| 7 | TRITON ® X-100 | 20 | 80/20 |
| 8 | C₈H₁₇C₆H₄SO₃Na (10) | 92 | 90/10 |
| 9 | C₁₂H₂₅C₆H₄SO₃Na (10) | 89 | 91/9 |

From Table 4, it can be seen that among various types of surfactants, in a system using an anionic surfactant (SDS or sodium benzenesulfonate having a long-chain alkyl group), the product was obtained with high yield and diastereoselectivity (Reactions 1 to 4, 8 and 9). On the other hand, when the surfactant was not added, it was revealed that, although diastereoselectivity was high, the reaction ratio was extremely low (Reaction 5). Further, even in a system in which a cationic surfactant, CTAB, or a nonionic surfactant, TRITON® X-100. is used, although a high diastereoselectivity was obtained, the reaction ratio was low (Reactions 6 and 7).

### Example 4

Then, specifying a FeCl₃-surfactant system as the catalyst, aldol reactions (reaction time: 12 to 24 hours) between various types of aldehydes and silicon enolates were studied. The results are shown in Table 5.

TABLE 5

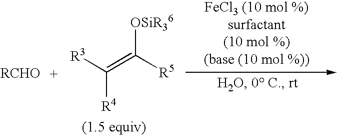

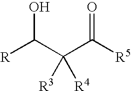

| | | Silicon | | | |
|---|---|---|---|---|---|
| Reaction | Aldehyde | enolate | Surfactant | Yield % | syn/anti |
| 1 | 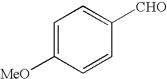 | 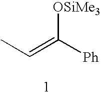 | C₁₂H₂₅C₅H₄SO₃Na | 86 | 91/9 |
| 2 | H₂C═CHCHO | 1 | C₁₂H₂₅C₆H₄SO₃Na | 66 | 87/13 |
| 3 | 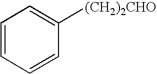 | 1 | C₁₂H₂₅C₆H₄SO₃Na | 76 | 89/11 |
| 4 | 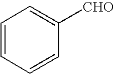 | 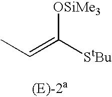 | SDS | 37 | 94/6 |
| 5^{b} | 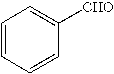 | (E)-2^{a} | SDS | 61 | 95/5 |
| 6^{b} | 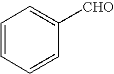 | 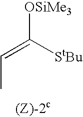 | SDS | 24 | 67/33 |
| 7^{b} | 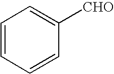 | 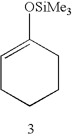 | SDS | 73 | 53/47 |
| 8^{b} | 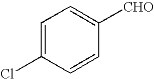 | 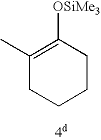 | SDS | 48 | 6/94^{e} |

^{a}E/Z = 95/5.
^{b}NaOH(10 mol %) was added.
^{c}E/Z = 2/98.
^{d}Mixture of regio isomers = 88/12.
^{e}The stereochemistry was assigned by analogy from the benzaldehyde adduct as follows.

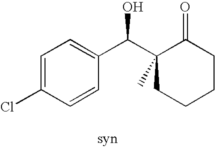

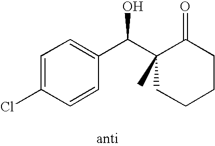

The silicon enolate (Compound 1) was reacted with an aromatic aldehyde, an a, p-unsaturated aldehyde and an aliphatic aldehyde, and the corresponding product was obtained with favorable yield and high diastereoselectivity (Reactions 1 to 3). Further, when the silicon enolate (Compound (E)-2) derived from S-tert-butyl thiopropionate was designated as a starting material, since the hydrolysis of Compound (E)-2 had rapidly progressed, the yield was decreased but a high diastereoselectivity was obtained (Reaction 4). Then, in order to suppress the hydrolysis of the silicon enolate, various types of bases were added to such systems as described above. Among a number of bases thus tested, NaOH was able to raise the reaction yield up to 61% (Reaction 5).

When Compound (Z)-2 and Compound 3 were used as silicon enolates, the diastereoselectivity of the reaction was between intermediate and low, while when Compound 4 was used, an extremely high diastereoselectivity was exhibited. In view of the above result together with the results of Reactions 1 to 5, it was confirmed that when there is a substituent in a cis-position of a silyl oxy group, there was a tendency for the compound to exhibit particularly high diastereoselectivity.

### INDUSTRIAL APPLICABILITY

As described above in detail, a method for performing an aldol reaction in water for obtaining a product with a high diastereoselectivity is provided by the invention.

Further, in the method for performing the aldol reaction according to the above-described first invention, by using FeCl₃ which is known to show strong Lewis acidity and a surfactant as the catalyst system, the aldol reaction in water between an aldehyde and a silicon enolate progresses with high diastereoselectivity and yield. Although FeCl₃ has conventionally been considered to be unsuited for use in water (Non-patent document 12), the present inventors have found that FeCl₃ can effectively act as a Lewis acid catalyst even in water. Further, since FeCl₃ is extremely low in cost, it is expected that such method for performing the aldol reaction in water as described above will be applicable to synthesis of a natural substance or the like on an industrial scale.

In the method for performing the aldol reaction in water according to any one of the above-described second to fifth inventions, by using an alkali metal salt of an alkyl sulfonic acid or an alkali metal salt of an alkylbenzenesulfonic acid, a product can be obtained with a particularly high yield and diastereoselectivity.

Further, in the method for performing the aldol reaction in water according to the above-described sixth or seventh inventions, by introducing a base, for example, sodium hydroxide, in the reaction system, hydrolysis of the silicon enolate is suppressed, to thereby enhance a reaction yield.

1. A method for performing an aldol reaction in water, being characterized in that an aldehyde represented by the following formula (I):
R—CHO (I)
(wherein R is a hydrocarbon group which may have a substituent), and a silicon enolate represented by the following formula (II): 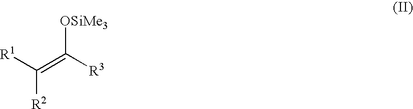
(wherein R¹ and R² are a hydrogen atom or an aliphatic hydrocarbon group, at least one of them being an aliphatic hydrocarbon group; and
R³ is a substituent selected from the group consisting of an aliphatic hydrocarbon group, an aromatic hydrocarbon group and a sulfur-containing substituent, wherein R² and R³ may be combined with each other to form a ring) are reacted with each other in water in the presence of FeCl₃ and a surfactant.

2. The method for performing the aldol reaction in water according to claim 1, wherein the surfactant is an alkali metal alkyl sulfate.

3. The method for performing the aldol reaction in water according to claim 2, wherein the surfactant is sodium dodecyl sulfate.

4. The method for performing the aldol reaction in water according to claim 1, wherein the surfactant is an alkali metal salt of an alkylbenzenesulfonic acid.

5. The method for performing the aldol reaction in water according to claim 4, wherein the surfactant is sodium octylbenzenesulfonate or sodium dodecylbenzenesulfonate.

6. The method for performing the aldol reaction in water according to claim 1, wherein the reaction is performed in the presence of a base.

7. The method for performing the aldol reaction in water according to claim 6, wherein the base is sodium hydroxide.

8. The method for performing the aldol reaction in water according to claim 2, wherein the reaction is performed in the presence of a base.

9. The method for performing the aldol reaction in water according to claim 3, wherein the reaction is performed in the presence of a base.

10. The method for performing the aldol reaction in water according to claim 4, wherein the reaction is performed in the presence of a base.

11. The method for performing the aldol reaction in water according to claim 5, wherein the reaction is performed in the presence of a base.

12. The method for performing the aldol reaction in water according to claim 8, wherein the base is sodium hydroxide.

13. The method for performing the aldol reaction in water according to claim 9, wherein the base is sodium hydroxide.

14. The method for performing the aldol reaction in water according to claim 10, wherein the base is sodium hydroxide.

15. The method for performing the aldol reaction in water according to claim 11, wherein the base is sodium hydroxide.
